(19) United States
(12) Patent Application Publication
(10) Pub. No.: US 2008/0188847 A1
(43) Pub. Date: Aug. 7, 2008

(54) BIOFEEDBACK

(75) Inventor: Morgan Lars Ake Gustavsson, Newton, MA (US)

Correspondence Address: PROSKAUER ROSE LLP, ONE INTERNATIONAL PLACE, BOSTON, MA, 02110, US

(73) Assignee: Candela Corporation, Wayland, MA (US)

(21) Appl. No.: 12/024,625

(22) Filed: Feb. 1, 2008

Related U.S. Application Data
(60) Provisional application No. 60/898,931, filed on Feb. 1, 2007.

(51) Int. Cl. A61B 18/18

(52) U.S. Cl. 606/33

(57) ABSTRACT

A process includes obtaining information relating to a biological tissue. The process also includes determining a treatment parameter from the information relating to the biological tissue. The process further includes delivering electromagnetic radiation having the treatment parameter to the biological tissue. An apparatus includes an interface capable of obtaining information relating to a biological tissue. The apparatus also includes a processor in communication with the interface, the processor capable of determining a treatment parameter from the information relating to the biological tissue. The apparatus further includes a source of electromagnetic radiation in communication with the processor, the source capable of delivering electromagnetic radiation having the treatment parameter to the biological tissue.

### CROSS-REFERENCE TO RELATED APPLICATION

This application claims the benefit of and priority to U.S. Provisional Application Ser. No. 60/898,931 filed Feb. 1, 2007, which is owned by the assignee of the instant application and the disclosure of which is incorporated herein by reference in its entirety.

### FIELD OF THE INVENTION

The invention relates generally to treating biological tissue. The invention relates more particularly to treating biological tissue using a beam of the electromagnetic radiation and biofeedback.

### BACKGROUND OF THE INVENTION

Electromagnetic radiation has a wide range of applications in treating biological tissue. For example, electromagnetic radiation has been employed in treatments as broad and varied as treatments for acne, erythema, fat, cellulite, oily skin, pigmented lesions, pores, scarring, vascular lesions, and wrinkles, as well as for skin rejuvenation, hair removal, and tattoo removal. However, electromagnetic radiation treatments can cause undesirable side effects such as burns, inflammatory reactions and pigmentary changes, which are routine and expected whenever the electromagnetic radiation is used to injure biological tissue. Furthermore, electromagnetic radiation treatments can occasionally cause acute side effects or permanent side effects, which can be a deterrent to individuals who otherwise desire treatment.

Different approaches to improving treatments and reducing side effects exist. For example, some methods for selectively ablating targeted biological material use real-time optical feedback control to measure photoemission from irradiated biological material and, based on the measured photoemission, adjust light pulse parameters to selectively affect targeted biological material. Other methods control the depth of ablation by feedback from the physiology of the skin, namely the infusion of blood into the area of excision when skin has been ablated to a sufficient depth to produce bleeding, and provide a feedback control mechanism which utilizes the optical characteristics such as the color, appearance and remittance of the definable skin layers to control the depth of ablation at each location. Still other methods for removing hairs from living skin involve the measurement of the color of the area of the skin where the hair is to be removed with a calorimeter to obtain a representative color value, employing the color value to select an optimum range of laser energy necessary to inactivate hair follicles in the area while minimizing inflammatory reactions, and directing laser energy of optimum range at the skin area to depilate such area.

### SUMMARY OF THE INVENTION

The invention, in one embodiment, features apparatus and methods for treating biological tissue using a beam of electromagnetic radiation and biofeedback. A user is prompted for information relating to a biological tissue to be treated. The user is provided with one or more treatment parameters for the electromagnetic radiation based on the information. The user is prompted to trigger a device capable of emitting the electromagnetic radiation to treat the biological tissue. The method can be used iteratively. The parameters for subsequent electromagnetic radiation emissions can be modulated based upon user input, which can be derived from the biological tissue's reaction to the preceding electromagnetic radiation emission. Also, the method can facilitate user operation by providing an automated user interface. One advantage of the invention can be increased or complete automation of treatment, which can increase the ease of use and reduce the potential for user error. Another advantage of the invention can be improved treatments of biological tissue that minimize blistering, burning, and non-therapeutic injury. In addition, using the invention, a doctor, a surgeon or a dermatologist is not needed to perform a treatment. A clinician, a nurse, or other less skilled practitioner at a spa, clinic, or other treatment facility can monitor the reaction of the skin and provide feedback to the treatment apparatus.

In various embodiments, the apparatus and methods can be used to treat acne, erythema, fat, cellulite, oily skin, pigmented lesions, pores, scarring, vascular lesions, and wrinkles, as well as for skin rejuvenation, hair removal, and tattoo removal. However, the treatment is not limited to the above indications, and can be used generally to treat biological tissue. For example, the biological tissue can be skin or lymphoid tissue. A beam of radiation can be delivered non-invasively to affect the biological tissue. In certain embodiments, the invention can be combined with other techniques known in the art to treat biological tissue.

In one aspect, the invention features a method. The method includes obtaining information relating to a biological tissue. The method also includes determining a treatment parameter from the information relating to the biological tissue. The method further includes delivering electromagnetic radiation having the treatment parameter to the biological tissue.

In another aspect, the invention features an apparatus. The apparatus includes an interface capable of obtaining information relating to a biological tissue. The apparatus also includes a processor in communication with the interface, the processor capable of determining a treatment parameter from the information relating to the biological tissue. The apparatus further includes a source of electromagnetic radiation in communication with the processor, the source capable of delivering electromagnetic radiation having the treatment parameter to the biological tissue.

In still another aspect, the invention features a method for treating a biological tissue with the electromagnetic radiation. The method includes prompting a user for information relating to the biological tissue to be treated. The method also includes providing the user with one or more treatment parameters for the electromagnetic radiation based on the provided information. The method further includes triggering a device capable of emitting the electromagnetic radiation to treat the biological tissue. A user can be prompted to trigger the device, or a user entering the information to the device can trigger the device.

In yet another aspect, the invention features an apparatus for treating biological tissue with the electromagnetic radiation. The apparatus includes a user interface, a processing unit, and a source of the electromagnetic radiation. The processing unit is coupled to the user interface and is configured to provide a signal to the user interface, to prompt a user for information relating to the biological tissue to be treated. The user interface is configured to provide a user input signal, including the information, to the processing unit. The source of the electromagnetic radiation is coupled to the processing unit. The processing unit is configured to provide a trigger signal to the source of the electromagnetic radiation, to cause the source to emit the electromagnetic radiation according to one or more treatment parameters based on the information of the user input signal.

In other examples, any of the aspects above, or any apparatus or method described herein, can include one or more of the following features.

In various embodiments, methods can include obtaining new information relating to biological tissue. The new information can relate to a result of delivering electromagnetic radiation having a treatment parameter to the biological tissue. Methods can also include determining a new treatment parameter from new information relating to biological tissue and delivering electromagnetic radiation having the new treatment parameter to the biological tissue.

In some embodiments, obtaining information can include prompting a user for information. An interface can be capable of prompting a user for information relating to biological tissue. Obtaining information can include employing a sensor capable of obtaining information relating to biological tissue. An interface can include a sensor capable of obtaining information relating to biological tissue.

In various embodiments, delivering electromagnetic radiation can include prompting a user to trigger an apparatus capable of delivering electromagnetic radiation. A processor can be capable of prompting a user to trigger an apparatus capable of delivering electromagnetic radiation. Delivering electromagnetic radiation can include triggering an apparatus capable of delivering electromagnetic radiation. A processor can be capable of triggering a source to deliver electromagnetic radiation. Delivering electromagnetic radiation can be capable of heating biological tissue to about a critical temperature.

In some embodiments, methods can include cooling at least a portion of the biological tissue. An apparatus can include a cooling system capable of cooling at least a portion of the biological tissue.

In certain embodiments, information relating to biological tissue can include at least one of a skin tan, skin type, lesion type, skin contrast, skin darkening, erythema, target size, target depth, target location, and reaction between the biological tissue and the electromagnetic radiation. A treatment parameter can be at least one of wavelength, power, fluence, duration, pulse or pulse train shape or width, spot size, and a parameter related to cooling of the skin.

In various embodiments, methods and apparatuses can be capable of treating at least one of a pigmented lesion, vascular lesion, fluorescent facial, skin tightening, scar, acne, hair, fat, cellulite, oily skin, pore, rejuvenation, and tattoo.

In some embodiments, methods can include at least one of sending and receiving data relating to a treatment through a communication network. A communication network interface can be capable of at least one of sending and receiving data relating to a treatment through a communication network. The invention can include at least one of storing and retrieving data relating to a treatment from a memory module.

In certain embodiments, methods include (i) prompting the user for information relating to at least one reaction between the biological tissue and the electromagnetic radiation, (ii) providing one or more further treatment parameters for the electromagnetic radiation based on the information provided by the user, and (iii) triggering the device to treat the biological tissue.

In various embodiments, methods include repeating (i) prompting the user for information relating to at least one reaction between the biological tissue and the electromagnetic radiation, (ii) providing one or more further treatment parameters for the electromagnetic radiation based on the information provided by the user, and (iii) triggering the device until treatment of the biological tissue is complete.

Other aspects and advantages of the invention can become apparent from the following drawings and description, all of which illustrate the principles of the invention, by way of example only.

### BRIEF DESCRIPTION OF THE DRAWINGS

FIG. 1 shows an exemplary cross-section of skin.

FIG. 2A shows an exemplary system for treating skin.

FIG. 2B shows an exemplary apparatus for treating biological tissue.

FIG. 3 shows another exemplary system for treating skin.

FIG. 4 shows a process of treating biological tissue using the electromagnetic radiation.

FIG. 5 shows a process of treating skin employing information relating to at least one reaction to adjust at least one treatment parameter.

FIG. 6 shows an exemplary network system including a local module and a remote module in communication through a communication network.

### DESCRIPTION OF THE INVENTION

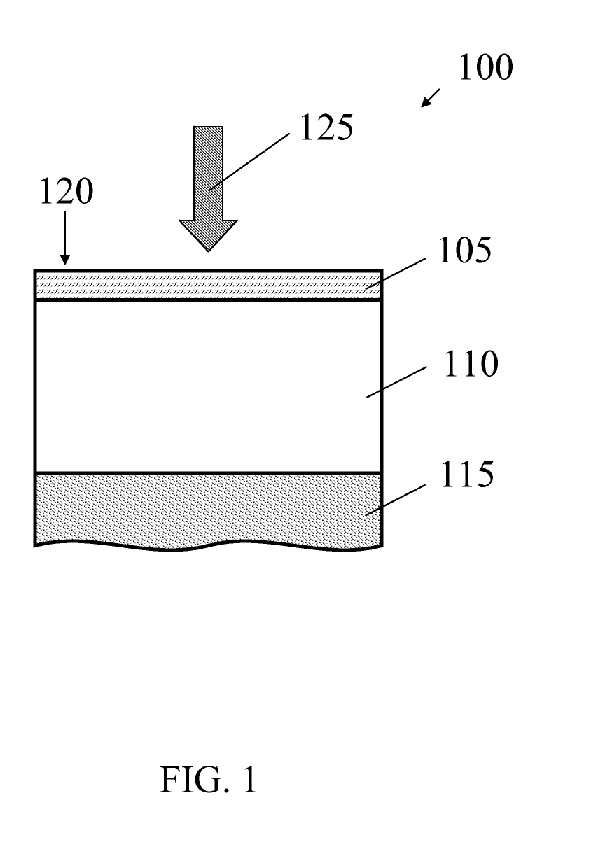

FIG. 1 shows an exemplary cross-section of skin 100 including a region of epidermis 105, a region of dermis 110, a region of subcutaneous tissue 115, and a surface of the skin 120. In one embodiment, the skin 100 can be a region of human skin. A beam of radiation 125 can be delivered to the skin 100 to treat at least a region of skin, including a region of epidermis 105 and/or a region of dermis 110. In various embodiments, a treatment skin treatment can be for acne, erythema, fat, cellulite, oily skin, pigmented lesions, pores, scarring, vascular lesions, and wrinkles, as well as for skin rejuvenation, hair removal, and tattoo removal. However, the treatment is not limited to the above indications, and can be used generally to treat biological tissue.

A therapeutic injury can be induced with the electromagnetic radiation in the visible to infrared spectral region. A wavelength of light that penetrates into at least a portion of biological tissue can be used. Chromophores can include blood (e.g., reduced and/or oxidized hemoglobin), collagen, melanin, feomelanin, fatty tissue, water, and porphryns. Light sources can include coherent light sources (e.g., lasers) and incoherent light source (e.g., lamps, light emitting diodes, and intense pulse light sources). The light source can be pulsed, continuous, or gated. In one embodiment, a light source can be coupled to a rigid waveguide or a flexible optical fiber or light guide, which can be introduced proximally to a target region of biological tissue. The light source can operate at a wavelength with depth of penetration into biological tissue that is less than the thickness of the target region of biological tissue.

In various embodiments, biological tissue in a target region is heated to a critical temperature to cause thermal injury. In certain embodiments, the critical temperature is below about 100° C. In other embodiments, the critical temperature is below about 95, 90, 85, 80, 75, 70, 65, 60, 55, or 50° C. In one embodiment, the critical temperature is the temperature associated with at least one of ablation, coagulation, necrosis, and acute thermal injury of biological tissue.

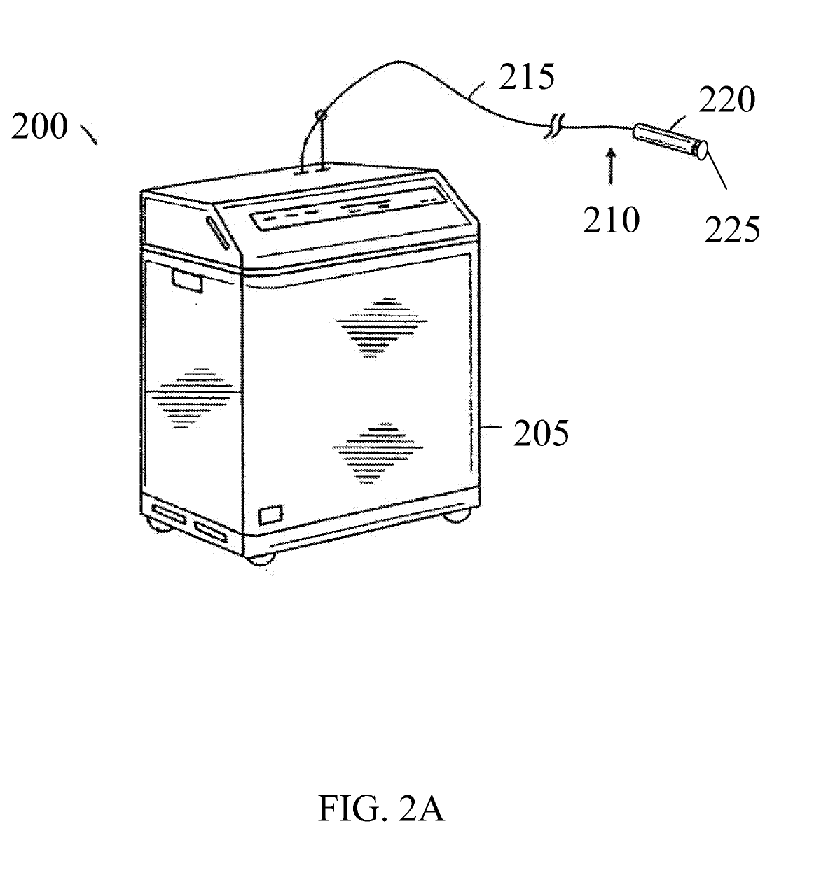

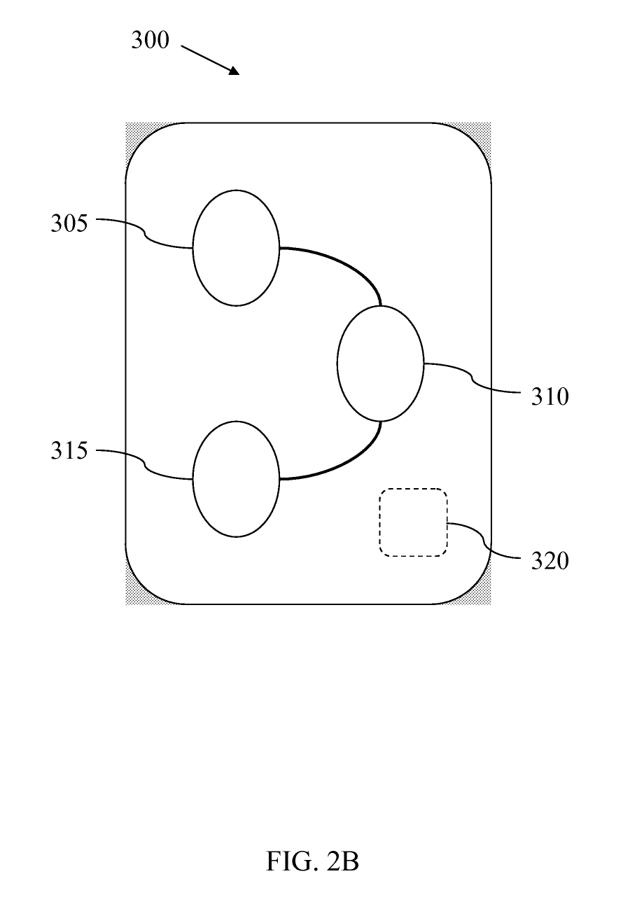

FIG. 2 shows an exemplary embodiment of a system 200 for treating biological tissue. The system 200 can be used to non-invasively deliver a beam of radiation to a target region of biological tissue. The system 200 includes a main unit 205 and a delivery system 210. In one embodiment, the main unit 205 includes an energy source that provides a beam of radiation directed via the delivery system 210 to a target area. In the illustrated embodiment, the delivery system 210 includes a fiber 215 having a circular cross-section and a handpiece 220. A beam of radiation can be delivered by the fiber 215 to the handpiece 220, which can include an optical system (e.g., an optic or system of optics) to direct the beam of radiation to the target area. A user can hold or manipulate the handpiece 220 to irradiate the target area. The handpiece 220 can be positioned in contact with a biological tissue surface, can be positioned adjacent a biological tissue surface, can be positioned proximate a biological tissue surface, can be positioned spaced from a biological tissue surface, or a combination of the aforementioned. In the embodiment shown, the handpiece 220 includes a spacer 225 to space the delivery system 210 from the biological tissue surface. In one embodiment, the spacer 225 can be a distance gauge, which can aid a practitioner with placement of the handpiece 220. In various embodiments, the system 200 can be an apparatus for treating biological tissue with the electromagnetic radiation.

FIG. 2B shows an exemplary apparatus 300 for treating biological tissue. The apparatus 300 can include a user interface 305, a processing unit 310, and a source of the electromagnetic radiation 315. In various embodiments the main unit 205 of the system can be, or include, an apparatus 300. The processing unit 310 is coupled to the user interface 305 and is configured to provide a signal to the user interface 305 prompting a user for information relating to biological tissue to be treated. The user interface 305 is configured to provide a user input signal, including the information, to the processing unit 310. The source of the electromagnetic radiation 315 is coupled to the processing unit 310. The processing unit 310 is configured to provide a trigger signal to the source of the electromagnetic radiation 315 to cause the source to emit the electromagnetic radiation, by the fiber 215 to the handpiece 220, according to one or more treatment parameters based on the user input signal.

In some embodiments, the apparatus 300 for treating biological tissue can include a memory module 320. The memory module 320 can store information relating to a treatment, including treatment parameters for a particular patient, tissue type, and/or body region. If a subsequent treatment is needed, the user can access the information stored in the memory module 320 and retrieve the information relating to a previous treatment. Information relating to previous treatment parameters can be used as initial parameters for a subsequent treatment.

After the electromagnetic radiation is delivered, the user interface 305 can prompt a user for input related to the biological tissue's reaction to the beam of electromagnetic radiation. Based on this input, a revised set of parameters can be determined for delivery of a subsequent beam of electromagnetic radiation.

In some embodiments, the user interface 305, processing unit 310, and source of the electromagnetic radiation 315 can be separate units. In one embodiment, the system 200 can include one or more sensors for detecting and/or measuring a reaction or a characteristic of the biological tissue. Output from a sensor can be directed to the user interface or processing unit 310, and can be used to modulate treatment parameters and/or properties of the emitted the electromagnetic radiation. Suitable sensors include heat cameras, IR sensors, spectrophotometers, image analysis cameras, CCDs, temperature sensors, erythema index sensors, and diode based color analyzers.

In various embodiments, the energy source 205 can be an incoherent light source (e.g., a lamp, a intense pulsed light system, or a fluorescent pulsed light system), a coherent light source (e.g., a laser), a solid state laser, a diode laser, a fiber coupled diode laser array, an optically combined diode laser array, and/or a high power semiconductor laser. In some embodiments, two or more sources can be used together to effect a treatment. For example, an incoherent source can be used to provide a first beam of radiation while a coherent source provides a second beam of radiation. The first and second beams of radiation can share a common wavelength or can have different wavelengths. In an embodiment using an incoherent light source or a coherent light source, the beam of radiation can be a pulsed beam, a scanned beam, or a gated continuous wave (CW) beam.

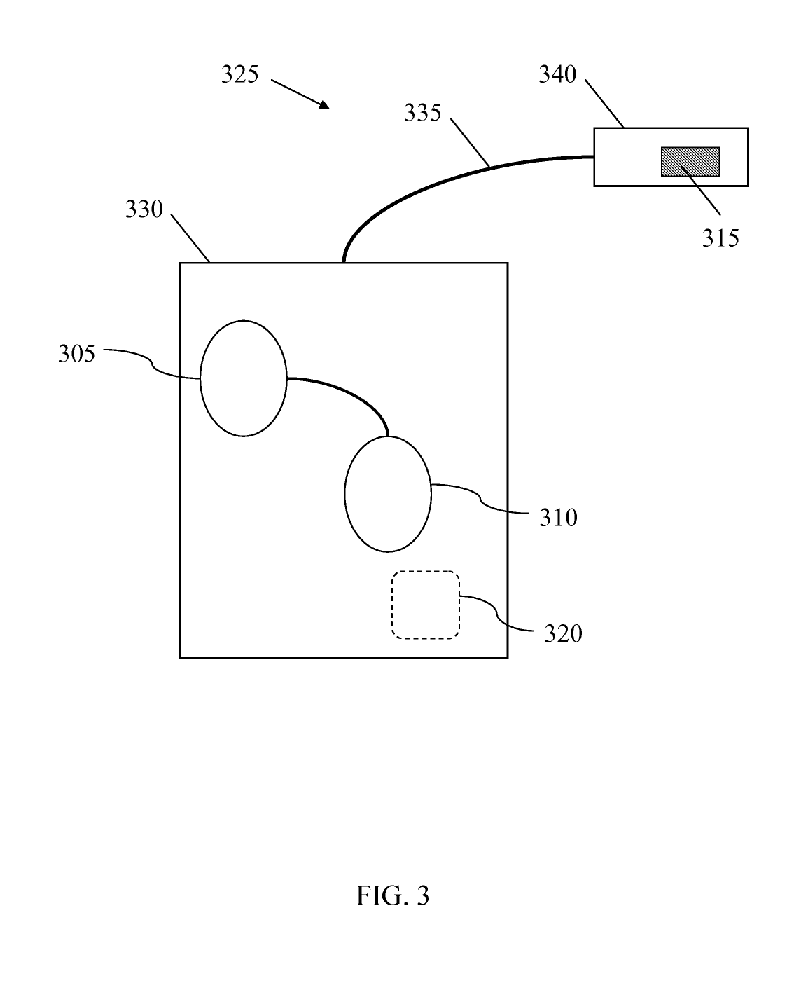

FIG. 3 shows a pulsed light system 325 including a base unit 330 and an umbilicus 335 connecting the base unit 330 to a handpiece 340. The base unit 330 can include the user interface 305 and the processing unit 310. The umbilicus 110 can include one or more conduits for communicating power, signal, fluid, and/or gas between the base unit 330 and the handpiece 340. The handpiece 340 include the source of the electromagnetic radiation 315. The handpiece 340 can include other components, such as filters and/or optics for delivering the electromagnetic radiation to biological tissue. Power can be used to drive the lamp, and signal can be used to control the output of the lamp (e.g., set, maintain, or control parameters of radiation being emitted from the lamp). The fluid and/or gas can be used to cool the source and/or a transparent or translucent member contacting the skin during treatment. Optionally, the base unit 330 can include memory module 320.

In various embodiments, the pulsed light system 325 can be a fluorescent pulsed light (FPL) or an intense pulsed light (IPL) system. For example, the system can be an OMNILIGHT™, NOVALIGHT™, or PLASMALITE™ system (by American Medical Bio Care of Newport Beach, Calif.). FPL technologies can utilize laser-dye impregnated polymer filters to convert unwanted energy from a xenon flashlamp into wavelengths that enhance the effectiveness of the intended applications. FPL technologies can be more energy efficient and can generate significantly less heat than comparative IPL systems. A FPL system can be adapted to operate as a multi-purpose treatment system by changing filters or handpieces to perform different procedures. For example, separate handpieces allow a practioner to perform tattoo removal and other vascular treatments.

In various embodiments, electromagnetic radiation delivered to biological tissue can be characterized by a pulse width between about 0.5 ms and about 100 s. In some embodiments, a pulse width is between about 5 ms and about 50 ms. In some embodiments, a pulse width is about 1, 2, 3, 4, 5, 10, 15, or 20 s.

Electromagnetic radiation delivered to biological tissue can be absorbed preferentially by a chromophore. For example, a chromophore can include at least one of hemoglobin in blood, melanin, porphyrin, exogenous pigment, and water in the skin. A chromophore can absorb the electromagnetic radiation preferentially over adjacent skin tissue. A spectrum characterizing electromagnetic radiation delivered to biological tissue can be matched to an absorption spectrum of a chromophore, including at least one of a whole blood, hemoglobin, reduced hemoglobin, and oxidized hemoglobin.

In various embodiments, the beam of radiation can have a wavelength between about 380 nm and about 2600 nm. For a given wavelength of radiation, a range of effective fluences can be approximated. Because wavelengths between about 380 nm and about 2600 nm are absorbed by water, and because biological tissue is about 70% water, an absorption coefficient of biological tissue can be approximated as 70% of an absorption coefficient of water. Because the absorption coefficient of water is a function of the wavelength of radiation, the desired fluence depends on the chosen wavelength of radiation. A fluence necessary to produce a desired damage depth can be approximated, for example, as the fluence that will raise the temperature to the critical temperature at the desired penetration depth, calculated as:

[3*μₐ*(μₐ+μₛ(1−g))]^{-0.5}

where μₐ, μₛ, and g are absorption coefficient, scattering coefficient, and the anisotropy factor of biological tissue, respectively.

Electromagnetic radiation delivered to biological tissue can be characterized by an energy density between about 0.1 J/cm² and about 500 J/cm². In various embodiments, electromagnetic radiation can be characterized by an energy density between about 1 and about 100 J/cm², about 2.5 J/cm² and about 60 J/cm², or about 2.5 J/cm² and about 12 J/cm². In certain embodiments, the energy density can be about 1, 5, 10, 50, 100, 150, 200, 250, 300, 350, 400, or 450 J/cm².

Electromagnetic radiation delivered to biological tissue can be characterized by a spot size between about 1 mm and about 20 mm. In various embodiments, a spot size can be up to about 1, 2, 3, 4, 5, or 10 mm in diameter.

Biological tissue can be treated with electromagnetic radiation and biofeedback. Information relating to biological tissue to be treated can be obtained, at least one treatment parameter for the electromagnetic radiation based on the information can be provided, and the electromagnetic radiation can be delivered to treat the biological tissue. The process can be documented as a hard copy or a computer data file, and can include data processing software.

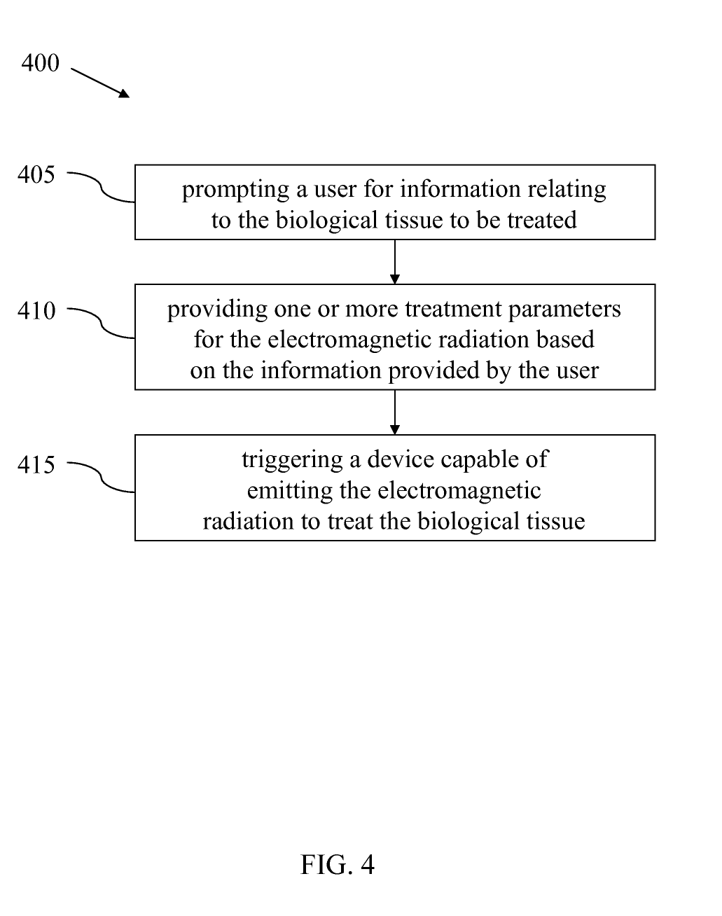

FIG. 4 shows a process 400 for treating a biological tissue using electromagnetic radiation. The process includes prompting a user for information relating to the biological tissue to be treated (step 405). For example, in various embodiments, the process 400 can include asking the user one or more questions and/or prompting the user to select or one or more options. The questions or options can include the desired treatment and/or one or more characteristics of the biological tissue. In some embodiments, the process 400 can include asking the user one or more questions about a related object such as a desired endpoint of treatment or a calibration standard.

The process 400 also includes providing one or more treatment parameters for the electromagnetic radiation based on the information provided by the user (step 410). In various embodiments, the one or more treatment parameters can include at least one property of the electromagnetic radiation. A property of the electromagnetic radiation can include one or more of the following: the portion of the electromagnetic spectrum, pulse or pulse train shape or width, application time, power, and/or fluence. In some embodiments, the one or more treatment parameters can include the duration, degree, and/or other parameters of cooling used in conjunction with the electromagnetic radiation.

The process 400 also includes triggering a device capable of emitting the electromagnetic radiation to treat the biological tissue (step 415). The device can be triggered by prompting the user, or by entering the information describing the reaction of the skin. The electromagnetic radiation is characterized by the one or more treatment parameters based on the information provided by the user. In some embodiments, one or more steps of the process 400 can be repeated or followed by one or more subsequent steps. In certain embodiments, process 400 can provide a complete course of treatment.

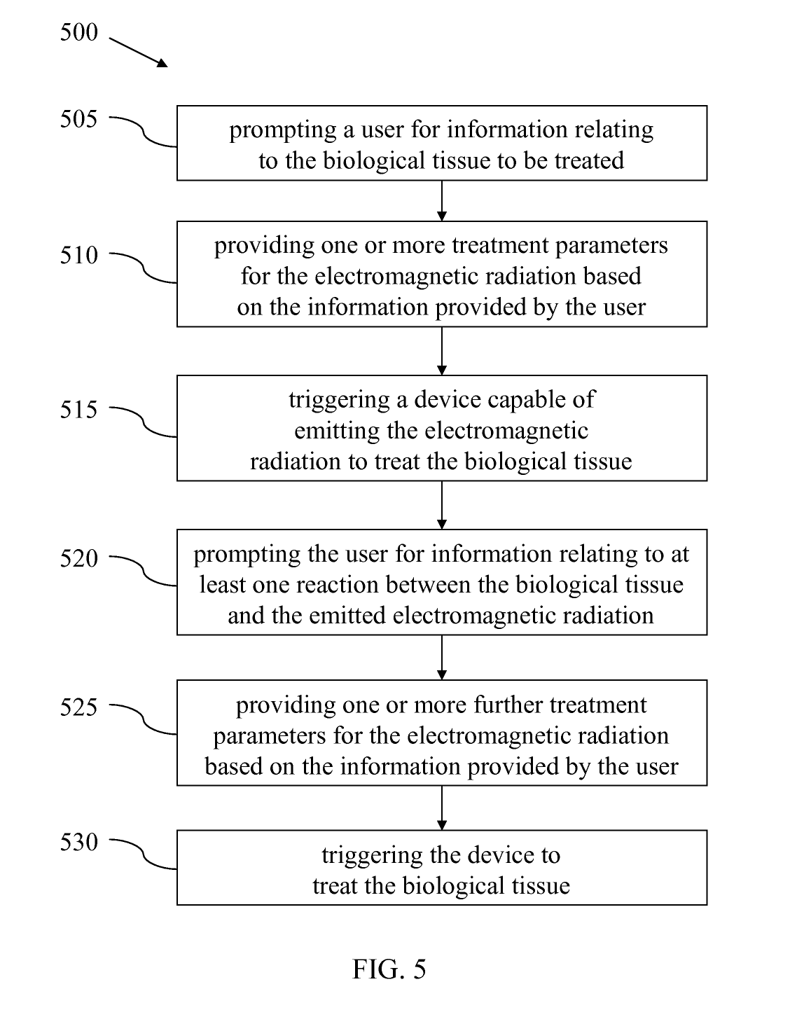

FIG. 5 shows a process 500 for treating a biological tissue using electromagnetic radiation. The process includes prompting a user for information relating to the biological tissue to be treated (step 505), providing one or more treatment parameters for the electromagnetic radiation based on the information provided by the user (step 510), and triggering a device capable of emitting the electromagnetic radiation to treat the biological tissue (step 515). In various embodiments, steps 505, 510, and 515 can include any of the aspects described in steps 405, 410, and 415.

The process 500 also includes prompting the user for information relating to at least one reaction between the biological tissue to the electromagnetic radiation (step 520). In various embodiments, the biological tissue can react and change color in response to the electromagnetic radiation. In some embodiments, a reaction (e.g., change in coloration or burning) can indicate that a treatment parameter is too intense (e.g., too high in fluence or too long in duration). In other embodiments, a reaction, or lack thereof, can indicate that a treatment parameter is not intense enough (e.g., too low in fluence or too short in duration). A reaction, or lack thereof, can also indicate that a treatment parameter is otherwise inappropriate or in need of modification. For example, a wavelength or other parameter may not sufficiently target the indication to be treated or the appropriate region of biological tissue. Furthermore, any reaction, or lack thereof, can indicate that the parameter(s) used are sufficient and need to be changed or only require a small adjustment.

The process 500 also includes providing one or more further treatment parameters for the electromagnetic radiation based on the information provided by the user (step 525). In various embodiments the information provided by the user can relate to the reaction caused by the electromagnetic radiation. The information can be biofeedback. The one or more further treatment parameters can mitigate treatment parameters that are too intense. The one or more further treatment parameters can also modulate treatment parameters that are not intense enough or are not sufficient to target the indication to be treated or appropriate region of biological tissue. The process 500 includes triggering the device to treat the biological tissue (step 530), employing the one or more further treatment parameters. One or more steps of the process 500 can be repeated, or followed by one or more subsequent steps. In certain embodiments, process 500 can provide a complete course of treatment.

The processes shown in FIGS. 4 and 5 can be iterative. For example, a process can include repeating (i) prompting the user for information relating to at least one reaction between the biological tissue and the electromagnetic radiation, (ii) providing one or more further treatment parameters for the electromagnetic radiation based on the information provided by the user, and (iii) prompting the user to trigger the device until treatment of the biological tissue is complete.

In various embodiments, information relating to biological tissue can be provided by a user and/or by one or more sensors. In some embodiments, the user can provide all of the information. In other embodiments, the one or more sensors can provide all of the information. In certain embodiments, the information can be provided by a combination of the user and the one or more sensors. Sensors can partially or completely automate treatment by providing information to set initial and/or subsequent treatment parameters. Suitable sensors are described above.

A cooling system can modulate the temperature in a region of biological tissue and/or minimize unwanted thermal injury to untargeted biological tissue. For example, the delivery system 200 shown in FIG. 2 can cool the biological tissue before, during, or after delivery of radiation, or a combination of the aforementioned. Cooling can include contact conduction cooling, evaporative spray cooling, convective air flow cooling, or a combination of the aforementioned. In one embodiment, the handpiece 220 includes a biological tissue contacting portion that can contact a region of biological tissue. The biological tissue contacting portion can include a sapphire or glass window and a fluid passage containing a cooling fluid. The cooling fluid can be a fluorocarbon type cooling fluid, which can be transparent to the radiation used. The cooling fluid can circulate through the fluid passage and past the window to cool the biological tissue.

A spray cooling device can use cryogen, water, or air as a coolant. In one embodiment, a dynamic cooling device (e.g., a DCD available from Candela Corporation) can cool the biological tissue. For example, the delivery system 200 shown in FIG. 2 can include tubing for delivering a cooling fluid to the handpiece 220. The tubing can be connected to a container of a low boiling point fluid, and the handpiece can include a valve for delivering a spurt of the fluid to the biological tissue. Heat can be extracted from the biological tissue by evaporative cooling of the low boiling point fluid. In one embodiment, the fluid is a non-toxic substance with high vapor pressure at normal body temperature, such as a Freon or tetrafluoroethane.

By cooling only a region of the target region or by cooling different regions of the target region to different extents, the degree of thermal injury of regions of the target region can be controlled.

### EXEMPLARY EMBODIMENTS

In various embodiments, one or more treatment parameters for the electromagnetic radiation can include wavelength, energy, time, and/or pulse or pulse train shape or width. In some embodiments, the tan of the skin can be used to adjust the treatment parameters (e.g., if the skin is not tanned, the power can be reduced). In certain embodiments, information relating to the biological tissue to be treated can include skin type. For example, a classification system, called the Fitzpatrick scale, has been established for grading the darkness of skin. The scale has six degrees of darkness referred to by type I through type VI.

In one embodiment, pigmented lesions can be treated. Information relating to the biological tissue to be treated can include skin type (e.g., I-VI), lesion type (e.g., deep or shallow), and contrast (e.g., dark, medium, or low). Information relating to at least one reaction between the biological tissue and the electromagnetic radiation can include information regarding darkening and/or erythema. For example: if no darkening results, then further treatment parameters can include increased energy and/or time; if darkening results after some minutes, then further treatment parameters can include increased energy and/or time; and if immediate darkening results, then further treatment parameters can include decreased energy. Also, for example: if erythema results only after a few minutes, then further treatment parameters can include increased energy; if immediate erythema results, then further treatment parameters can include decreased energy; if general erythema results, then further treatment parameters can include further decreased energy; and if erythema results only around the lesion, then further treatment parameters can include still further decreased energy.

In another embodiment, vascular lesions can be treated. Information relating to the biological tissue to be treated can include skin type (e.g., I-VI) and lesion type (e.g., telangiectasia, rosacea, polokiloderma, port wine stain, cherry angioma, or spider naevus), as well as the size, depth, and/or location of the lesion. Information relating to at least one reaction between the biological tissue and the electromagnetic radiation can include information regarding vessel clearing and/or erythema. For example: if no vessel clearing results, then further treatment parameters can include increased energy and/or time; however, if is vessel clearing results, then further treatment parameters can include increasing energy. Also, for example: if no erythema results or if erythema results only after a few minutes, then further treatment parameters can include increased energy; if erythema results only around the lesion, then further treatment parameters can include a smaller energy increase; if immediate erythema results, then further treatment parameters can include a still smaller energy increase; and if general erythema results, then further treatment parameters can include an even smaller energy increase. In each of these cases, the erythema adjustment can include increasing time.

In still another embodiment, fluorescent facial treatments can be provided. Information relating to at least one reaction between the biological tissue and the electromagnetic radiation can include information regarding pigment darkening and/or erythema. For example: if no pigment darkening results, then further treatment parameters can include increased energy and/or time; if pigment darkening results after some minutes, then further treatment parameters can include increased energy; and if pigment darkening results immediately, then further treatment parameters can be the same as preceding treatment parameters.

In still yet another embodiment, skin tightening treatments can be provided. Information relating to at least one reaction between the biological tissue and the electromagnetic radiation can include information regarding erythema. For example: if no erythema results, if erythema results only after a few minutes, or if only some erythema results, then further treatment parameters can be the same as preceding treatment parameters; if erythema results immediately, then further treatment parameters can include decreased energy; and if general erythema results, then further treatment parameters can include further decreased energy.

In one embodiment, scar treatments can be provided. Information relating to the biological tissue to be treated can include skin type (e.g., I-VI) and scar color (e.g., red/brown or pale hypertrophic). Information relating to at least one reaction between the biological tissue and the electromagnetic radiation can include information regarding erythema. For example: if no erythema results, then further treatment parameters can be the same as preceding treatment parameters; if erythema results after some minutes, then further treatment parameters can include decreased energy; if some erythema results, then further treatment parameters can include further decreased energy; and if general or immediate erythema results, then further treatment parameters can include still further decreased energy.

In another embodiment, acne treatments can be provided. Information relating to the biological tissue to be treated can include skin type (e.g., I-VI) and acne type (inflammatory, post-inflammatory, or acne scarring). Information relating to at least one reaction between the biological tissue and the electromagnetic radiation can include information regarding erythema.

In still another embodiment, hair removal treatments can be provided. Information relating to the biological tissue to be treated can include skin type (e.g., I-VI), hair color (e.g., black, brown, blond, or grey) and hair type (e.g., course, medium, thin, or vellus). Information relating to at least one reaction between the biological tissue and the electromagnetic radiation can include information regarding edema and/or erythema. For example: if no edema results, then further treatments parameters can include increased energy; if perifollicular edema results, then further treatment parameters can be the same as preceding treatment parameters; and if general edema results, then further treatment parameters can include decreased energy. Also, for example: if no erythema results, if perifollicular erythema results, or if erythema results after some minutes, then further treatment parameters can be the same as preceding treatment parameters; if immediate erythema results, then further treatment parameters can include decreased energy; and if general erythema results, then further treatment parameters can include further decreased energy.

In other embodiments, fat, cellulite, oily skin, pore, skin rejuvenation, and tattoo removal treatments can be provided.

In various embodiments, a system 200 for treating biological tissue and/or an apparatus 300 for treating biological tissue can send and/or receive information to and from a remote site through a network. For example, treatment parameters can be stored remotely and accessed when a particular reaction is identified by a user. This permits an outside agency to change, update, or add treatment parameters as new parameters are determined, e.g., by academic research or clinical studies.

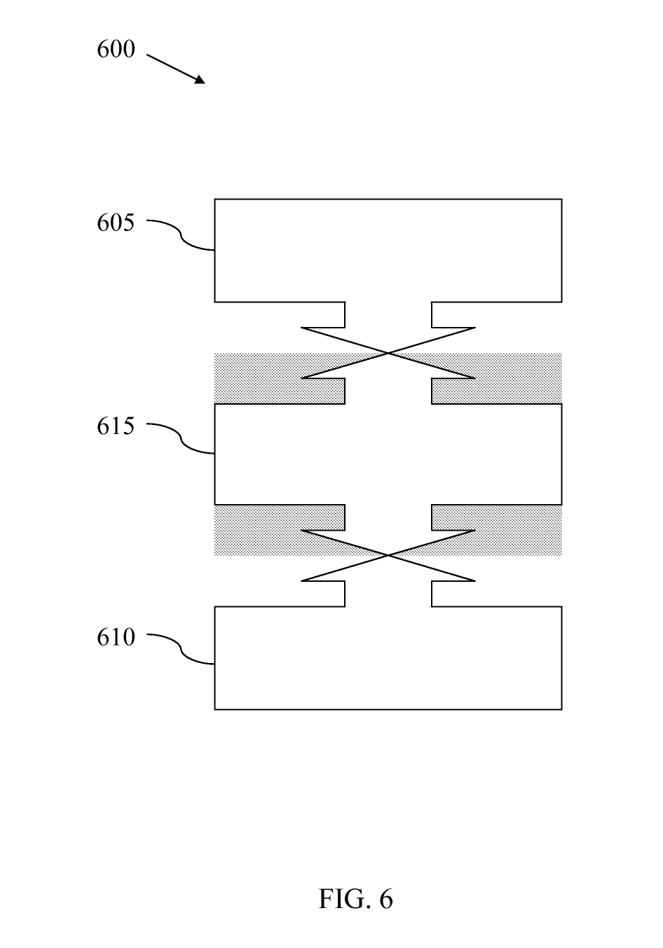

FIG. 6 shows an exemplary network system 600 including a local 605 module and a remote 610 module, which are in communication through a communication network 615. The local 605 module is configured to provide a treatment, and can include a system 200 for treating biological tissue and/or an apparatus 300 for treating biological tissue. In various embodiments, the local 605 module can include one or more computers, servers, firewalls, databases, or other network devices to process, send, and/or receive information through the communication network 615. The remote 610 module can include one or more computers, servers, firewalls, databases, or other network devices to process, send, and/or receive information through the communication network 615. The communication network 615 can be a private company network, for example an intranet, or a public network, for example the internet.

In various embodiments the local 605 module can transmit information to the remote 610 module. For example, the local 605 module can transmit information relating to the biological tissue to be treated and/or information relating to at least one reaction between the biological tissue and the electromagnetic radiation. Based upon the information, the remote 610 module can provide one or more treatment parameters and/or one or more further treatment parameters based upon the information provided. The remote 610 module can calculate treatment parameters and/or retrieve treatment parameters from a database. In some embodiments, the remote 610 module can collect, store, and/or analyze information from multiple treatments by a user and/or multiple users.

The above-described techniques can be implemented in digital electronic circuitry, or in computer hardware, firmware, software, or in combinations of them. The implementation can be as a computer program product, i.e., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

Process steps can be performed by one or more programmable processors executing a computer program to perform functions of the invention by operating on input data and generating output. Process steps can also be performed by, and apparatus can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Data transmission and instructions can also occur over a communications network. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

The terms “module” and “function,” as used herein, mean, but are not limited to, a software or hardware component which performs certain tasks. A module may advantageously be configured to reside on addressable storage medium and configured to execute on one or more processors. A module may be fully or partially implemented with a general purpose integrated circuit (IC), FPGA or ASIC. Thus, a module may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and modules may be combined into fewer components and modules or further separated into additional components and modules. Additionally, the components and modules may advantageously be implemented on many different platforms, including computers, computer servers, data communications infrastructure equipment such as application-enabled switches or routers, or telecommunications infrastructure equipment, such as public or private telephone switches or private branch exchanges (PBX). In any of these cases, implementation may be achieved either by writing applications that are native to the chosen platform, or by interfacing the platform to one or more external application engines.

To provide for interaction with a user, the above described techniques can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer (e.g., interact with a user interface element). Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The above described techniques can be implemented in a distributed computing system that includes a back-end component, e.g., as a data server, and/or a middleware component, e.g., an application server, and/or a front-end component, e.g., a client computer having a graphical user interface and/or a Web browser through which a user can interact with an example implementation, or any combination of such back-end, middleware, or front-end components. The components of the system can be interconnected by any form or medium of digital data communication, e.g., a communication network. Examples of communication networks include a local area network (“LAN”) and a wide area network (“WAN”), e.g., the Internet, and include both wired and wireless networks. Communication networks can also all or a portion of the PSTN, for example, a portion owned by a specific carrier.

The computing system can include clients and servers. A client and server are generally remote from each other and typically interact through a communication network. The relationship of client and server arises by virtue of computer programs running on the respective computers and having a client-server relationship to each other.

While the invention has been particularly shown and described with reference to specific embodiments, it should be understood by those skilled in the art that various changes in form and detail may be made therein without departing from the spirit and scope of the invention as defined by the appended claims.

1. A method comprising:
obtaining information relating to a biological tissue;
determining a treatment parameter from the information relating to the biological tissue; and
delivering electromagnetic radiation having the treatment parameter to the biological tissue.

2. The method of claim 1 further comprising:
obtaining new information relating to the biological tissue, the new information relating to a result of delivering electromagnetic radiation having the treatment parameter to the biological tissue;
determining a new treatment parameter from the new information relating to the biological tissue; and
delivering electromagnetic radiation having the new treatment parameter to the biological tissue.

3. The method of claim 1 wherein obtaining information includes prompting a user for information.

4. The method of claim 1 wherein obtaining information includes employing a sensor capable of obtaining information.

5. The method of claim 1 wherein delivering electromagnetic radiation includes prompting a user to trigger an apparatus capable of delivering electromagnetic radiation.

6. The method of claim 1 wherein delivering electromagnetic radiation includes triggering an apparatus capable of delivering electromagnetic radiation.

7. The method of claim 1 wherein delivering electromagnetic radiation is capable of heating the biological tissue to about a critical temperature.

8. The method of claim 1 further comprising cooling at least a portion of the biological tissue.

9. The method of claim 1 wherein information relating to the biological tissue comprises at least one of a skin tan, skin type, lesion type, skin contrast, skin darkening, erythema, target size, target depth, target location, and reaction between the biological tissue and the electromagnetic radiation.

10. The method of claim 1 wherein the treatment parameter comprises at least one of wavelength, power, fluence, duration, pulse or pulse train shape or width, spot size, and a parameter related to cooling of the skin.

11. The method of claim 1 wherein the method is a treatment for at least one of a pigmented lesion, vascular lesion, fluorescent facial, skin tightening, scar, acne, hair, fat, cellulite, oily skin, pore, rejuvenation, and tattoo.

12. The method of claim 1 further comprising at least one of sending and receiving data relating to a treatment through a communication network.

13. The method of claim 1 further comprising at least one of storing and retrieving data relating to a treatment from a memory module.

14. An apparatus comprising:
an interface capable of obtaining information relating to a biological tissue;
a processor in communication with the interface, the processor capable of determining a treatment parameter from the information relating to the biological tissue; and
a source of electromagnetic radiation in communication with the processor, the source capable of delivering electromagnetic radiation having the treatment parameter to the biological tissue.

15. The apparatus of claim 14 wherein the interface is capable of prompting a user for information relating to the biological tissue.

16. The apparatus of claim 14 wherein the interface comprises a sensor capable of obtaining information relating to the biological tissue.

17. The apparatus of claim 14 wherein the processor is capable of prompting a user to trigger the source to deliver the electromagnetic radiation.

18. The apparatus of claim 14 wherein the processor is capable of triggering the source to deliver the electromagnetic radiation.

19. The apparatus of claim 14 wherein delivering the electromagnetic radiation is capable of heating at least a portion of the biological tissue to about a critical temperature.

20. The apparatus of claim 14 further comprising a cooling system capable of cooling at least a portion of the biological tissue.

21. The apparatus of claim 14 wherein information relating to the biological tissue comprises at least one of a skin tan, skin type, lesion type, skin contrast, skin darkening, erythema, target size, target depth, target location, and reaction between the biological tissue and the electromagnetic radiation.

22. The apparatus of claim 14 wherein the treatment parameter comprises at least one of wavelength, power, fluence, duration, pulse or pulse train shape or width, spot size, and a parameter related to cooling of the skin.

23. The apparatus of claim 14 wherein the apparatus is capable of treating at least one of a pigmented lesion, vascular lesion, fluorescent facial, skin tightening, scar, acne, hair, fat, cellulite, oily skin, pore, rejuvenation, and tattoo.

24. The apparatus of claim 14 further comprising a communication network interface capable of at least one of sending and receiving data relating to a treatment through a communication network.

25. The apparatus of claim 14 further comprising a memory module capable of at least one of storing and retrieving data relating to a treatment.

26. An apparatus for treating a biological tissue with electromagnetic radiation, comprising:
a user interface;
a processing unit coupled to the user interface, the processing unit configured to provide a signal to the user interface prompting a user for information relating to the biological tissue to be treated and the user interface configured to provide a user input signal, including the information, to the processing unit; and
a source of the electromagnetic radiation coupled to the processing unit, the processing unit further configured to provide a trigger signal to the source of the electromagnetic radiation to cause the source to emit the electromagnetic radiation according to one or more treatment parameters based on the user input signal.
